(19) United States
(12) Patent Application Publication
(10) Pub. No.: US 2024/0138463 A1
(43) Pub. Date: May 2, 2024

(54) INFANT FORMULAS CONTAINING HUMAN BREAST MILK PROTEINS

(71) Applicant: MILK CARE CO., INC., Framingham, MA (US)

(72) Inventor: Wendel DE OLIVEIRA AFONSO, Lynn (OM)

(21) Appl. No.: 18/547,567

(22) PCT Filed: Feb. 24, 2022
(86) PCT No.: PCT/US2022/017607

§ 371 Date: Aug. 23, 2023

Related U.S. Application Data
(60) Provisional application No. 63/152,932, filed on Feb. 24, 2021.

(51) Int. Cl. A23L 33/00; A23L 33/12; A23L 33/175; A23L 33/19

(52) U.S. Cl. CPC A23L 33/40; A23L 33/12; A23L 33/175; A23L 33/19

(57) ABSTRACT

The present invention in various aspects and embodiments provides infant formulas containing human milk proteins, optionally in combination with non-human animal milk proteins such as cow's milk and/or goat's milk protein, and/or vegetable proteins. In some embodiments, the sole source of milk proteins in the formula is recombinant or extracted human milk protein. In various embodiments, the infant formula is particularly beneficial for newborns diagnosed with cow's milk protein allergy, or other sensitive babies, who are unable to consume dairy products. Accordingly, the invention in other aspects provides methods for providing nutrition to newborns or infants diagnosed with cow's milk protein allergy, allergic proctocolitis, or otherwise infants that are intolerant of formulas comprising cow's milk protein, hydrolyzed formulas, or amino acid-based formulas.

### BACKGROUND

According to World Health Organization (WHO) breastfeeding is the best way to provide infants with the nutrients they need for healthy growth and development. Colostrum, produced at the end of pregnancy, is recommended by WHO as the perfect food for the newborn. Moreover, exclusive breastfeeding is recommended up to 6 months of age, with continued breastfeeding along with appropriate complementary foods up to two years of age or beyond. Breast milk is the only food naturally “designed” for babies. Breast milk is generally recognized as allowing for the best growth and development of young infants, the least dietary-related problems in their early life, and less dietary-related issues later in life such as cardiovascular disease and metabolic syndrome.

The commercial introduction of infant formula was of great success in overcoming the high mortality rates faced until the nineteenth century among infants who could not be breastfed. More than a century of research, developments and trials have made infant formula a safer and nutritionally sound food. The goal of the vast majority of infant formula producers is to mimic the composition and/or to match the functionality of breast milk as closely as possible.

Before the invention of infant formula, there were basically two options: wet nursing (i.e., a baby being fed by a woman other than its mother) or dry nursing alternative feeding based on mammals' milk and predigested and wheat-containing foods. Wet nursing was by far the safest option of the two. Where breast milk was not accessible to the newborn, survival rates neared 0%. Major breakthroughs in the nineteenth century paved the way for the development of safe and nutritionally sound infant formula and germ-free feeding bottles. In 1865, the first commercial infant formula was developed, providing the basic foundation of the present-day formula for newborns. Nowadays, access to breastfeeding no longer implies a matter of life and death.

Health outcomes differ substantially for mothers and infants who formula feed compared with those who breastfeed infants. For infants, not being breastfed is associated with an increased incidence of infectious morbidity, including otitis media, gastroenteritis, and pneumonia, as well as elevated risks of childhood obesity, type 1 and type 2 diabetes, leukemia, and sudden infant death syndrome. Among premature infants, not receiving breast milk is associated with an increased risk of necrotizing enterocolitis. Infant feeding is an important modifiable risk factor for disease for both mothers and infants.

The American College of Obstetricians and Gynecologists therefore recommends six months of exclusive breastfeeding for all infants. The American Academy of Pediatrics and the American Academy of Family Physicians similarly recommend exclusive breastfeeding for the first six months of life, continuing at least through the infant's first birthday, and as long thereafter as is mutually desired. The World Health Organization recommends at least two years of breastfeeding for all infants.

In the United States, breastfeeding durations fall far short of these guidelines. In 2005, 74.2% of US infants were breastfed at least once after delivery, but only 31.5% were exclusively breastfed at age three months, and just 11.9% were exclusively breastfed at age six months. Public health campaigns and medical literature have traditionally described the “benefits of breastfeeding,” comparing health outcomes among breastfed infants against a reference group of formula-fed infants.

Due to the high rates of formula-fed babies in the contemporary age, and since cow's milk is the main ingredient of these products, several consequences have been reported including allergies and intolerances. Cow's milk protein allergy (CMPA) is the most common food allergy in childhood. The prevalence of allergic diseases has been dramatically rising in the United States and other developed nations in recent decades. Food protein-induced allergic proctocolitis (AP) is among the earliest and most common food allergic diseases of infancy, yet its pathophysiology is not well understood. Generally, the national data do not report non-IgE-mediated food allergies or describe the subset of non-IgE-mediated allergy exclusively triggered by milk. AP typically presents in early infancy with mucous and blood in the stool (either hematochezia or guaiac-positive stools) and non-specific symptoms of fussiness, difficulty feeding, and gastroesophageal reflux. Eosinophilic inflammation on histology of rectal biopsy was also associated with patients who presented AP symptoms. Symptoms typically resolve with dietary antigen restriction, and cow's milk is the most common trigger. Symptoms suggestive of AP affect upwards of 10-15% of infants (Martin et al., 2020).

Most children present with CMPA at age less than 1 year and therefore may require a hypoallergenic formula in the absence of breast milk. Several alternatives to cow's milk and cow's milk-based formulas are available, including extensively hydrolyzed formulas (EHF), which are hypoallergenic cow's proteins, and amino acid-based formulas (AAF). For most children with a CMPA, an EHF will be sufficient for symptom resolution but there is a subset of children with CMPA where an AAF may be indicated. For example, an AAF might be indicated when symptoms are not fully resolved on EHF; the infant shows signs of slow growth/failure to thrive; multiple food eliminations; or the patient exhibits signs of severe complex gastrointestinal food allergies, eosinophilic esophagitis, food protein-induced enterocolitis syndrome, or severe eczema and symptoms while breast-feeding. In addition, patients who end up on an AAF often present with multisystem involvement, requiring multiple food eliminations and fall within the more severe spectrum of gastrointestinal allergies. In eosinophilic esophagitis, all current recommendations support the use of an AAF as first-line approach, and in children with anaphylaxis, an AAF is recommended because of the potential risk for a severe reaction.

AAF and EHF are considered equally efficacious at relieving the symptoms of CMPA in confirmed or suspected cases. Some clinical benefit has been reported from the use of AAF, in both symptoms and growth in infants and children with CMPA and who fail to tolerate EHF.

Nevertheless, future studies need to assess the impact of long-term AAF use, and it may be that the introduction of other proteins during weaning is necessary. Further, prolonged absence of whole dietary protein after weaning may impair immunological maturation and the development of tolerance.

Moreover, in a study focused primarily on the flavor and the relative palatability of hydrolyzed and amino acid-based formulas, frequent complaints were reported that children reject infant formulas for treatment of CMPA due to the bad taste (Pedrosa Delgado et al., 2006; Miraglia Del Giudice et al., 2015). It has also been proposed that a child's refusal to accept an EHF (e.g., due to the bitter taste) is reason enough to switch to another hypoallergenic option (Vandenplas et al., 2014). Considering that in such situations the infant formula is the baby's only food source, dietary issues related to bad taste/smell are crucial to implementation and success of the clinical intervention. A statistically significant correlation between peptide weight, reflecting the degree of hydrolysis of each formula, and the scores obtained for taste, texture, and overall palatability, have been reported (Pedrosa et al., 2006).

Beyond the palatability, none of the EHFs are completely free of allergens (Dupont et al., 2015). Adverse effects of EHFs have been reported, including vomiting/spitting up and watery/bloody diarrhea (Inuo et al., 2018), and rare serious reactions such as cases of anaphylactic shock and apparent life-threatening events (Cantani and Micera, 2015; Bocquet et al., 2019). Considering that these formulas are given for prolonged periods, failure to respond to an EHP formula can result in impaired growth and sustained allergy symptoms (Vanderhoof et al., 2016). Regarding AAF, although most children tolerate the formula, especially in severe cases (Koletzko et al., 2012), some long term, adverse effects have been described, such as hypophosphatemia, fractures, rickets, and other bone diseases (Gonzalez Ballesteros et al., 2017; Akhtar Ali S. et al., 2019).

It is an object of the invention to provide an infant formula without the use of substantial non-human animal protein, and which avoids the unpleasant odor, taste, and/or adverse reactions associated with current products often used for infants with cow's milk allergy and sensitive babies.

### DETAILED DESCRIPTION OF THE INVENTION

The present invention in various aspects and embodiments provides infant formulas containing recombinant human milk proteins and/or proteins extracted from human milk, optionally in combination with non-human animal milk proteins such as cow's milk and/or goat's milk protein, and/or plant or vegetable proteins. In exemplary embodiments, the infant formula further comprises one or more of casein, casein hydrolysate, whey protein isolate (WPI), whey protein concentrate (WPC), whey protein-lipid concentrate (WPLC), whey protein hydrolysate (WPH), and free amino acids. In some embodiments, the sole source of milk proteins in the formula is recombinant and/or extracted human milk protein. In various embodiments, the infant formula is particularly beneficial for newborns diagnosed with cow's milk protein allergy, or other sensitive babies, who are unable to consume dairy products. These patients currently use high-cost formulas containing only free amino acids or hydrolyzed proteins instead of intact cow's milk proteins. These elemental infant formulas have a bitter taste and an unpleasant odor and generate a high rate of adverse reactions. Accordingly, the invention in other aspects provides methods for providing nutrition to newborns or infants diagnosed with cow's milk protein allergy, allergic proctocolitis, or otherwise intolerant of formulas comprising cow's milk protein, hydrolyzed formulas, or amino acid-based formulas.

Infant formulas available on the market can be characterized as powdered milk obtained from mammals of different species (primarily cow, but sometimes goat), rebalanced by the addition of macro and micronutrients such as vitamins, minerals, lipids and carbohydrates. Up to 17% of babies experience side effects from the use of non-human milk in formula, ranging from “softer” effects such as gas and discomfort to more extreme side effects such as reflux, diarrhea, pain, inability to thrive and allergy to cow's milk proteins. The current formulas used to treat babies with allergy to cow's milk use extensively hydrolyzed cow's proteins or amino acids as the base. These ingredients not only bring a bitter taste and an unpleasant odor to the formulas but also generate adverse reactions, such as osmotic diarrhea, vomiting and nausea.

In accordance with embodiments of the invention, baby formula containing recombinant and/or extracted human breast milk proteins as a substantial ingredient, instead of cow's or goat milk proteins, will not only substantially reduce side effects associated with regular formulas that are largely caused by proteins from cow's milk, but also will provide a superior treatment of cow's milk protein allergy in sensitive newborns and infants.

Human breast milk has over 1,600 distinct proteins and other major and minor components, and thus the whole human milk cannot be identically replicated at a molecular level. However, in accordance with this disclosure, an infant formula can be prepared with less than about fifteen human breast milk proteins, or less than about ten or less than about five human breast milk proteins, in order to be a substantial substitute for human breast milk, including for patients exhibiting signs of sensitivity, CMPA, or AP. In some embodiments, a single human breast milk protein can be employed as a suitable substitute for human breast milk. In some embodiments, the infant formula may contain proteins extracted from human milk, such as WPI, WPC, or WPLC from human milk. The infant formula will reduce various side effects of current formula's for CMPA including incidence of gas, reflux, diarrhea, nausea, vomiting and/or discomfort. Alternatively, or in addition, the present invention provides infant formulas that avoid the bitter taste and unpleasant odor associated with current alternatives, including alternatives currently available for sensitive patients with CMPA or AP.

In some embodiments, the infant formula is prepared with about 15 recombinant human breast milk proteins or less, or about 12 recombinant human breast proteins or less, or about 10 recombinant human breast milk proteins or less, or about 8 recombinant human breast milk proteins or less, or about 5 recombinant human breast milk proteins or less. In some embodiments, the formula contains 1, 2, 3, or 4 distinct recombinant human breast milk proteins. In some embodiments, the formula comprises from 2 to 7 recombinant human breast milk proteins, or from 3 to 7 or from 3 to 5 recombinant human breast milk proteins. In some embodiments, the infant formula comprises recombinant human breast milk casein(s) (e.g., A2 β casein), optionally with from 1 to 5 (e.g., 1, 2, 3, 4, or 5) recombinant whey proteins. In some embodiments, the infant formula contains one or more whey proteins, without any caseins. In some embodiments, the formula contains only one recombinant human breast milk protein. The infant formula is stable in dry form and can be easily mixed and solubilized with water. In some embodiments, the infant formula is provided constituted with water.

In some embodiments, the predominant or sole source of proteins in the formula is recombinant human breast milk proteins, optionally with proteins extracted from human breast milk. In other embodiments, the predominant or sole source of proteins in the formula is human milk proteins extracted from human milk.

In various embodiments, the infant formula meets the international nutritional standards and may comprise oils, carbohydrates, amino acids, vitamins, minerals, and nitrogenous source. In some embodiments, the formula further comprises fibers, and optionally probiotics and/or prebiotics. The formula in various embodiments is similar to breast milk in terms of nutrition and does not have a bitter taste and/or unpleasant smell.

The human breast milk proteins may be produced by a recombinant technology in microorganisms, plant cells, insect cells or mammalian cells, and purified for incorporation into the infant formula. Exemplary fermentation systems include yeast expression systems such as Pichia pastoris, Yarrowia hpolytica, and Saccharomyces cerevisiae. Other suitable expression systems include bacterial expression systems such as E. coli. In some embodiments, the recombinant human breast milk proteins are expressed and purified from a single recombinant host strain, or alternatively, are expressed and purified from different host strains. In some embodiments, the recombinant human breast milk proteins are prepared by batch fermentation, with the human breast milk proteins secreted into and purified from the fermentation media. Purification systems can comprise one or more of filtration, crystallization, precipitation, and chromatography (including affinity or size chromatography, for example).

Alternatively or in addition, human breast milk proteins (including one or more of the human breast milk proteins described herein) can be extracted from human milk, and incorporated into the infant formula. For example, in some embodiments, whey proteins are extracted. For example, in some embodiments, extracted proteins comprise one or more of α-lactalbumin, osteopontin, and lysozyme, among others. For example, extracted proteins can include proteins having an apparent molecular weight (i.e., based on filtration) of at least about 5 kDa, or at least about 10 kDa. In some embodiments, the extracted proteins comprise proteins having an apparent molecular weight (based on filtration) of less than about 150 kDa, or less than about 100 kDa, or less than about 75 kDa, or less than about 50 kDa, or less than about 40 kDa, or less than about 25 kDa. Protein may be extracted from human milk by substantial removal of casein (e.g., by acid precipitation), followed by one or more steps of filtration, to recover proteins of the desired molecular weight range (e.g., about 10 to about 100 kDa, about 10 to about 75 kDa, about 10 to about 50 kDa, or about 10 to about kDa). In these or other embodiments, caseins are extracted from human milk using known methods, and optionally are partially hydrolyzed. Extracted proteins can be dried, powdered, and used as a supplement for infant formulas. In some embodiments, extracted proteins are further purified by other means, such as precipitation, crystallization, and chromatography (e.g., size or affinity chromatography). Levels of major proteins in the extracted sample can be determined by known techniques if desired. Dry protein from extraction can be used as an infant formula ingredient, along with lactose, vegetable oils, vitamins and minerals premix. The result is superior to commercial hypoallergenic infant formulas (casein hydrolysate and amino acid-based formulas) regarding flavor, odor, color and general appearance. The infant formula containing extracted human breast milk proteins have better sensory characteristics and the physical-chemical analysis demonstrate the adequacy of the final product to nutritional recommendation.

In various embodiments, the recombinant human breast milk proteins are selected from α-lactalbumin, β-caseins, serum albumin, lactoferrin, κ-Casein, osteopontin, lysozyme, immunoglobulins (IgA), and Epidermal Growth Factors (EGF). In various embodiments, the invention substantially replicates the colostrum or transitional or mature breast milk protein compositions, in the sense that the formula also avoids the bitter taste and unpleasant smell of other formulas, as well as gastrointestinal side effects induced by these formulas. The composition may optionally be supplemented with essential and/or non-essential amino acids. In some embodiments, the composition is supplemented with other proteins sources (which are optionally hydrolyzed or partially hydrolyzed) such as plant protein, yeast protein, and animal protein.

In some embodiments, the composition comprises one or more human recombinant whey proteins, and particularly α-lactalbumin and/or albumin as the predominant protein source, optionally with one or more proteins selected from lactoferrin, osteopontin, lysozyme, immunoglobulins (IgA), and Epidermal Growth Factors (EGF). In these embodiments, the composition does not contain any casein proteins, but is supplemented with one or more amino acids to provide the necessary nutrients.

For example, the composition in some embodiments contains only recombinant human α-lactalbumin, and is supplemented with methionine. Since α-lactalbumin provides low levels of methionine, this amino acid is supplemented in the absence of casein. In some embodiments, phenylalanine and/or valine are further supplemented, which have higher levels in casein than whey proteins such as α-lactalbumin. In accordance with these embodiments, an infant formula can be prepared that better mimics the taste and smell of human milk, remarkably with only a single recombinant protein. In such embodiments, the challenges of producing recombinant casein proteins with phosphorylation and glycosylation states suitable for forming stable micelles is avoided. In various embodiments, the infant formula contains from about 5 to about 15 g of whey protein (e.g., α-lactalbumin) per 100 g of formula on a dry basis (e.g., about 7 g to about 12 g per 100 g of formula), with from about 10 milligrams to about 100 milligrams of each of L-methionine, L-phenylalanine, and L-valine (on a dry basis). In some embodiments, the formula contains from about 20 milligrams to about 80 milligrams of each of L-methionine (e.g., about 38 mg), L-phenylalanine (about 38 mg), and L-valine (e.g., about 67 mg) (on a dry basis).

Alternatively, the composition in some embodiments contains only recombinant human serum albumin as a protein source and is supplemented with isoleucine. Since albumin provides low levels of isoleucine, this amino acid is supplemented in the absence of casein. In some embodiments, tryptophan is further supplemented. In still other embodiments, threonine and/or methionine are further supplemented, which have higher levels in casein than whey proteins such as albumin. In accordance with these embodiments, an infant formula can be prepared that better mimics the taste and smell of human milk, remarkably with only a single recombinant protein. In such embodiments, the challenges of producing recombinant casein proteins with phosphorylation and glycosylation states suitable for forming stable micelles is avoided. In various embodiments, the infant formula contains from about 5 to about 15 g of whey protein (e.g., albumin) per 100 g of formula on a dry basis (e.g., about 7 g to about 12 g per 100 g of formula), with from about 200 to 500 mg of L-isoleucine (e.g., about 357 mg), and with from about 50 milligrams to about 200 milligrams of L-tryptophan (e.g., about 133 mg), and 10 to 100 mg each of L-methionine (e.g., about 29 mg) and L-threonine (e.g., about 32 mg) (on a dry basis).

In some embodiments, the human protein and amino acid components are tailored for the age of the infant. Human milk and its key components, including proteins, change continuously over time. Consequently, narrowing the gap between breast milk and infant formula requires a greater understanding how protein quality and quantity in human milk changes. Human milk is a source of essential nutrients for infants and therefore its composition at the different stages of lactation comprises different nutrient contents. The stage of lactation can be divided into three stages depending on the time: colostrum (first few days after birth), transitional, and mature milk. As lactation progresses the chemical composition of human milk changes as a result of physiological and external factors. The levels of casein and whey proteins change profoundly in the early stages of lactation; whey protein concentration is very high, and casein is low during the initiation of lactation (Guo 2021). As lactation progresses, casein synthesis in the mammary gland and milk production increase, while the concentration of whey proteins decreases, in part due to a larger volume of milk produced. Therefore, the ratio of whey:casein is not constant, but fluctuates between about 80:20 in early lactation, to about 50:50 in late lactation (Lönnerdal 2003). Because the amino acid contents of whey proteins and casein differ, milk amino acid content also changes as infants mature.

The protein content in human milk also changes during lactation, ranging from 1.4 to 2.0 g/100 mL during early lactation, 1.1 to 1.3 g/100 mL by 3 to 6 months of lactation, to 0.7 to 0.8 g/100 mL after 6 months. In the early postpartum period (days 1-4), immunoglobulin A (IgA) and lactoferrin (LF) are the two major proteins in human colostrum, although there is variability between individuals. For casein concentration, there is a dramatic increase during the first week postpartum, after which it remains relatively stable during days 6-28. After that, the concentration shows an upward trend. The casein content is about 22.5%-45.8% of total protein throughout the process of lactation. In various embodiments of this disclosure, the infant formula provides from about 7 g to about 20 g of protein per 100 mg of formula (on a dry basis), or from about 7 g to about 15 g of protein per 100 g of formula (on a dry basis), or from about 9 to about 15 g of protein per 100 g of formula (on a dry basis).

Overall, the median protein content in milk expressed between 16 and 30 days after delivery is 24% lower compared with true protein in milk expressed 0 to 5 days after delivery (1.57 g/100 mL vs. 2.06 g/100 mL). By 90 to 360 days, the protein content in human breast milk is about 47% lower compared to 0 to 5 days after delivery (1.10 g/100 mL).

α-Lactalbumin (LA) is the major protein in human milk, serving an important nutritional function. The LA concentration constantly decreases during lactation. Other proteins increase during lactation, which can help infants strengthen their immune system. For example, there is a strong increase in absolute and relative concentrations of lactoferrin and particularly of lysozyme (LZ) in days 50-84, which could take effect as anti-infectious agents in the passive protection of infants during mature lactation.

Lipids are important nutrients in human milk, providing approximately 50% energy for infants. However, it is the most variable component of human milk and is markedly influenced by lactational stage. In general, the human milk fat content is found to be significantly increased from around 3.5%-4.5% during lactation, and it continues to increase even after 12 and 18 months of lactation (Sinkiewicz-Darol et al. 2021; Yuan et al. 2021). Colostrum milk fat constituted a higher content in PUFAs (ω-6, and long-chain ω-6 and ω-3) than transitional and mature milk fats, with the corresponding lower content of saturated fatty acids (SFA) in its sn-2 position. Lower percentages of monounsaturated fatty acids, arachidonic acid (AA), and C22:5 ω-3 fatty acid are found in transitional and mature milk than in colostrum (Zou et al. 2012). Percentages of saturated fatty acids and C18:0 were higher in transitional and mature milk than in colostrum. There was an increase in the amount of C18:3 ω-3 during the course of lactation, while percentages of C16:0, C20:3 ω-6, DHA, total ω-6 and ω-3 LCPUFAs decreased as lactation progressed (Sala-Vila et al. 2005).

In some embodiments, sources of fats are provided to avoid tastes or smells that may be refused by the infant. For example, in some embodiments, oils with more neutral odors and tastes are employed, while oils that have “beany” or “nutty” flavors or odors are avoided. For example, in various embodiments, vegetable oils selected from canola oil, sunflower oil, safflower oil, coconut, and corn, or a combination thereof are employed. In various embodiments, oils such as soybean, walnut, or sesame oil are avoided. In some embodiments, the fats include SN2 palmitate.

Carbohydrates are the most stable components in human milk and the lactose concentration increases slightly during the course of lactation, ranging from 5.5 to 7.3 g/100 mL, depending on lactation duration (Perrin et al. 2017; Sinkiewicz-Darol et al. 2021). As for the human milk oligosaccharides (HMOs), a slight gradual increase is observed (Perrin et al. 2017).

Therefore, infant formulas are adjusted to the nutritional variations observed during the lactation period, in relation to the concentration and proportion of macronutrients, such as protein content and whey:casein ratio, fat content and fatty acid profile, as carbohydrate content, comprising lactose and HMOs concentration. These variations help meet the nutritional need for infants, which vary throughout the first months of life.

In various embodiments, the formula contains at least one, or at least two, or at least three, or at least four, or at least five, or at least six, or at least seven, or all recombinant human breast milk proteins selected from a α-lactalbumin, β-casein, serum albumin, lactoferrin, κ-casein, osteopontin, lysozyme, Immunoglobulin (e.g., IgA) and Epidermal Growth Factor (EGF). In some embodiments, the selected human breast milk protein(s) are in combination with other sources of protein such as extracted human breast milk proteins, cow's milk protein, goat's milk protein, whey protein, as well as casein and/or albumin from other sources. In some embodiments, the selected human breast milk proteins are supplemented with plant proteins such as soy protein, pea protein, rice protein, or other plant sources, and at a level that does not substantially impact the flavor and/or odor. In some embodiments, the formula is supplemented with other hypoallergenic protein sources, such as hydrolyzed animal (e.g., cow or goat) or plant proteins and amino acids. In some embodiments, the formula does not contain any non-human animal protein, plant protein, or hydrolyzed protein.

In some embodiments, the infant formula comprises cow's milk characterized as A2 milk (more digestible cow's milk) enriched with human breast milk proteins to feed sensitive infants or babies with cow's milk protein allergy symptoms. Regular cow's milk contains both A1 and A2 beta-casein, but A2 milk contains only A2 beta-casein. In some embodiments, the infant formula comprises hydrolyzed/digested cow's milk proteins enriched with human β-caseins classified as A2 β-caseins, to feed infants with cow's milk protein allergy symptoms.

Caseins are phosphoproteins commonly found in mammalian milk and comprise nearly 80% of the protein in cow's milk, and from about 20% to about 45% of the protein in human milk. As a food source, casein supplies amino acids, carbohydrates, and two essential elements, calcium and phosphorus. Key caseins in human milk include β-casein and κ-casein. The αs1 casein subunit is present in very low concentrations in human milk, unlike in cow's milk. When β-casein is digested, smaller casein phosphopeptides and caseomorphins are formed. Negatively charged casein phosphopeptides can chelate Ca2+ and may facilitate calcium absorption. Although it has been proposed that bovine casein phosphopeptides do not enhance calcium absorption in adults, the presence of such peptides in infants may help to keep calcium in solution and thereby improve net calcium absorption. The presence of casein phosphopeptides in human milk may explain, in part, the more effective uptake of calcium from breast milk than from formula. Casein phosphopeptides may also contribute to the absorption of zinc and other divalent cations. Caseomorphins have structures similar to opioid peptides and may thus affect infant sleep wake patterns and psychomotor development. β-casein may also exhibit antimicrobial activity towards Haemophilus influenza and streptococci. κ-casein inhibits bacterial adhesion, including the adhesion of Helicobacter pylon. In fact, H. pylori is less common in breast-fed than in formula-fed infants. This may result from the structural similarity between the glycans of casein and the surface-exposed carbohydrates of cells in the mucosa of the gastrointestinal tract, suggesting that these glycans may act as soluble “decoys” for pathogens. Studies also indicate that caseins may exhibit immunomodulatory activity by regulating chemotaxis and ameliorating inflammation.

κ-casein, a highly glycosylated human milk protein, provides defense against infection. κ-casein inhibits adherence of Helicobacter pylori to human gastric mucous, and of Streptococcus pneumoniae and Hemophilus influenzae to human respiratory-tract epithelial cells. It also promotes the growth of Bifidobacterium bifidum, an acid-producing anaerobe that reduces the growth of intestinal pathogenic microorganisms in breastfed infants, due to the presence of the C-terminus proteolysis product of κ-casein.

Casein has relatively little tertiary structure and is relatively hydrophobic, making it poorly soluble in water. It is found in milk as a suspension of particles. The casein core structure is rich in hydrophobic amino acids. The bitterness taste and sulfureted smell arises during hydrolysis due to the presence of low molecular weight peptides composed mainly of hydrophobic amino acids while salty off flavor is due to the pH production adjustments.

An attractive property of the casein molecule is its ability to form a gel or clot in the stomach, which makes it very efficient in nutrient supply. The clot is able to provide a sustained slow release of amino acids into the blood stream, sometimes lasting for several hours. Hydrolyzed casein can be responsible for a bitter taste and refusal by infants of compositions containing hydrolyzed casein.

Accordingly, in some embodiments, the human protein component of the infant formula contains from about 1% to about 100% recombinant or extracted human casein(s), such as β caseins and/or κ caseins. In some embodiments, the human protein component of the infant formula contains from about 5% to about 75% recombinant or extracted human casein(s), such as β caseins and/or κ caseins. In some embodiments, the human protein component of the infant formula contains from about 5% to about 50% recombinant or extracted human casein(s), such as β caseins and/or κ caseins. In some embodiments, the human protein component of the infant formula contains from about 20% to about 50% recombinant or extracted human casein(s) or from about 20% to about 40% recombinant or extracted human caseins, such as β caseins and/or κ caseins. Alternatively, the recombinant or extracted caseins are from about 40% to about 100% of the total human protein content, or from about 50% to about 100% of the total human protein content, or from about 70% to about 100% of the total human protein content, or from about 80% to about 100% of the total human protein content and may include β caseins and/or κ caseins.

In some embodiments, the casein in the infant formula will form micelles with other components (e.g., other proteins, surfactants such as mono- and diglycerides and oils) upon mixing with water. The micelles generally have a diameter of less than about 100 nm.

Lactalbumin is the albumin contained in milk and obtained from whey. Lactalbumin is found in the milk of many mammals. There are α- and β-lactalbumins; both are contained in milk. α-lactalbumin is a protein that regulates the production of lactose in the milk of almost all mammalian species. In primates, α-lactalbumin expression is upregulated in response to the hormone prolactin and increases the production of lactose. α-lactalbumin has an approximate molecular weight of 14 kDa. α-lactalbumin can play an important role as a protein source, in presence or absence of caseins.

Accordingly, in some embodiments, the human protein component of the infant formula is from about 1% to about 100% recombinant or extracted human α-lactalbumin, or in some embodiments, from about 5% to about 75% recombinant or extracted human α-lactalbumin, or from about 5% to about 50% recombinant or extracted human α-lactalbumin, or about 5% to about 40% recombinant or extracted human α-lactalbumin, or from about 5% to about 30% recombinant or extracted human α-lactalbumin. In some embodiments, the human protein component of the infant formula is from about 50% to about 100% recombinant or extracted human α-lactalbumin, or from about 50% to about 90% recombinant or extracted human α-lactalbumin, or from about 50% to about 75% recombinant or extracted human α-lactalbumin. In some embodiments, human α-lactalbumin (recombinant or extracted) is present in the formula at from about 1% to about 15%, such as about 5% to about 10% of the total protein content. When whey proteins such as α-lactalbumin are used alone without caseins, caseins can be replaced with free amino acids. In various embodiments, methionine is supplemented, optionally with phenylalanine and valine. Other essential and/or non-essential amino acids can also be supplemented. Essential amino acids are Histidine, Isoleucine, Leucine, Lysine, Methionine, Cysteine, Phenylalanine, Threonine, Tyrosine, Tryptophan, and Valine.

Lactoferrin is a multifunctional protein of the transferrin family. Lactoferrin is a globular glycoprotein with a molecular mass of about 80 kDa that is widely represented in various secretory fluids, including milk. Lactoferrin has antimicrobial activity (bactericidal and fungicidal activity) and is part of the innate defense, mainly at mucosal surfaces. Accordingly, in some embodiments, the human protein component of the infant formula is from about 1% to about 100% human lactoferrin (either recombinant or extracted), or in some embodiments, from about 1% to about 75% human lactoferrin, or from about 1% to about 50% human lactoferrin, or from about 1% to about 40% human lactoferrin, or from about 1% to about 10% human lactoferrin. In some embodiments, the human protein component of the infant formula is from about 10% to about 100% human lactoferrin, or from about 20% to about 90% human lactoferrin, or from about 50% to about 75% human lactoferrin. In some embodiments, human lactoferrin (recombinant or extracted) is present in the formula at from about 0.1% to about 10%, such as about 1% to about 7% of the total protein content.

Osteopontin is a multifunctional protein present in human milk, and is present in higher concentration in early lactation, and it is related to better immune outcomes. Osteopontin has an approximate molecular weight of 33 kDa. Accordingly, in some embodiments, the human protein component of the infant formula is from about 1% to about 100% osteopontin (either recombinant or extracted), or in some embodiments, from about 1% to about 75% osteopontin, or from about 1% to about 50% osteopontin, or from about 1% to about 40% osteopontin, or from about 1% to about 10% osteopontin. In some embodiments, the human protein component of the infant formula is from about 10% to about 100% osteopontin (either recombinant or extracted), or from about 20% to about 90% osteopontin, or from about 50% to about 75% osteopontin. In certain embodiments, the protein component of the infant formula is from about 0.1% to about 1.0% human osteopontin (either recombinant or extracted), or in some embodiments, from about 0.2% to about 2.0% human osteopontin by weight of the total protein content.

Serum albumin is also a component of human breast milk. Albumin functions primarily as a carrier protein for steroids, fatty acids, and thyroid hormones in the blood. Human serum albumin has an approximate molecular weight of 66.5 kDa. Accordingly, in some embodiments, the human protein component of the infant formula is from about 10% to about 100% human albumin (either recombinant or other source), or in some embodiments, from about 10% to about 75% human albumin, or from about 10% to about 50% human albumin, or about 10% and 40% human albumin, or from about 10% and about 30% human albumin. In some embodiments, the human protein component of the infant formula is from about 50% to about 100% human albumin (either recombinant or other source), or from about 50% to about 90% human albumin, or from about 50% to about 75% human albumin.

Lysozyme is an antimicrobial enzyme produced by animals that forms part of the innate immune system. Lysozyme is a glycoside hydrolase that catalyzes the hydrolysis of 1,4-beta-linkages between N-acetylmuramic acid and N-acetyl-D-glucosamine residues in peptidoglycan, which is the major component of gram-positive bacterial cell wall. This hydrolysis in turn compromises the integrity of bacterial cell walls causing lysis of the bacteria. Lysozyme is abundant in secretions including human milk and has a molecular weight of about 15 kDa. Accordingly, in some embodiments, the human protein component of the infant formula is from about 1% to about 100% recombinant or extracted human lysozyme, or in some embodiments, from about 1% to about 75% recombinant or extracted human lysozyme, or from about 1% to about 50% recombinant or extracted human lysozyme, or from about 10% to about 40% recombinant or extracted human lysozyme, or from about 10% to about 30% recombinant or extracted human lysozyme. In some embodiments, the human protein component of the infant formula is from about 50% to about 100% recombinant or extracted human lysozyme, or from about 50% to about 90% recombinant or extracted human lysozyme, or from about 50% to about 75% recombinant or extracted human lysozyme.

Immunoglobulin A (IgA) is an antibody that plays a crucial role in the immune function of mucous membranes. Secretory IgA (sIgA) is the main immunoglobulin found in colostrum. sIgA can also inhibit inflammatory effects of other immunoglobulins and is a poor activator of the complement system. In accordance with these embodiments, non-specific IgA is believed to provide a protective function for the infant, and/or can be an important component of the composition in terms of smell and/or taste. Accordingly, the human protein component of the infant formula is from about 5% to about 100% recombinant or extracted human IgA, or in some embodiments, from about 5% to about 75% recombinant or extracted human IgA, or from about 5% to about 50% recombinant or extracted human IgA, or from about 5% to about 40% recombinant or extracted human IgA, or from about 10% to about 30% recombinant or extracted human IgA. In some embodiments, the human protein component of the infant formula is from about 50% to about 100% recombinant or extracted human IgA, or from about 50% to about 90% recombinant or extracted human IgA, or from about 50% to about 75% recombinant or extracted human IgA.

Soluble growth factors found in breast milk include epidermal growth factor (EGF), which activates the EGF receptor (EGFR). EGF can be beneficial in protecting the newborn intestines from early inflammatory insult. EGF is one of the major peptide growth factors present both in colostrum and human milk. Human milk EGF levels are highest in the first days after parturition and then gradually decrease during the first 2 week of lactation. EGF is not found in commercial infant formulas. EGF can support repair processes in injured intestinal mucosa. The EGF component of the formula can be recombinant or extracted from human milk. In various embodiments, the human protein component of the infant formula contains about 0.001% to about 10% of recombinant human EGF, such as in some embodiments, from about 0.01% to about 5% recombinant human EGF, or from about 0.01% to about 1% recombinant human EGF, or from about 0.01% to about 0.1% recombinant human EGF.

In some embodiments, the infant formula contains recombinant human milk proteins selected from (or consisting of) β-casein, α-lactalbumin, and lactoferrin, and osteopontin and optionally EGF. In some embodiments, the dry infant formula (per 100 g of dry formula) contains from about 5 g to about 15 g human recombinant or extracted human α-lactalbumin. In some embodiments, the dry infant formula (per 100 g of dry formula) contains about 3 g to about 10 g human recombinant or extracted human α-lactalbumin and about 1 g to about 10 g of human recombinant or extracted β-casein. In some embodiments, the dry infant formula (per 100 g of dry formula) contains from 2 g to about 5 g human recombinant or extracted β-caseins, about 3 g to about 8 g human recombinant or extracted α-lactalbumin, and about 0.1 g to about 3 g human recombinant or extracted lactoferrin, and optionally about 0.01 to about 1 g of human recombinant or extracted osteopontin. The formula may further optionally contain about 0.1 g to about 3 g EGF (which can be recombinant EGF). In various embodiments, the infant formula has a protein content of from about 5% to about 25% protein (i.e., 5 to 25 grams of protein per 100 grams of dry formula), or in some embodiments, from about 8% to about 20% protein.

In certain aspects, the invention provides a set of infant formulas (e.g., 2, 3, or 4 infant formulas) with decreasing whey:casein ratio. The whey and casein proteins can be selected from those as described above and herein. A first infant formula, intended for use in the first 1-2 weeks (e.g., about 10 days), contains a whey:casein ratio of about 75:25. A second infant formula, intended for use after the first formula and up to about the first month (e.g., from about day 11 to about day 30), contains a whey:casein ratio of about 63:37. A third infant formula, intended for use after the second formula and up to about 3 months (e.g., from about day 31 to about day 90), contains a whey:casein ratio of about 55:45. A fourth infant formula, intended for use after the third formula, contains a whey:casein ratio of about 50:50.

In these or other embodiments, the infant formula has a carbohydrate component of from about 30% to about 70% (i.e., 30 to 70 g per 100 grams of dry formula), such as from about 40% to about 65%, or about 50% to about 60%. Accordingly, in some embodiments, the carbohydrate component may comprise one or more of: lactose, maltose, sucrose, glucose, maltodextrins, glucose syrup, pre-cooked starch, corn syrup solids, rice syrup solids, galactooligosaccharide (GOS), fructooligosaccharide (FOS), and human milk oligosaccharides (HMO), and any combinations thereof. In some embodiments, the predominant carbohydrate source is lactose.

HMOs constitute a heterogeneous mixture of glycans that vary per individual. The amounts of HMOs in human milk is dependent on the stage of lactation and varies from around 20.9 g/L in colostrum to 12.9 g/L in mature milk. They have multiple functions which include support of the growth of beneficial bacteria, influencing microbiota composition, anti-pathogenic effects, immune-modulating effects, stimulating intestine barrier functions and preventing infection and supporting immunity. Commercially available HMOs include 2′-FL (2′-Fucosyllactose) and lacto-N-neotetrose (LNnT) (or mixture thereof), and which may be (each or together) about 0.5% to about 2.0% by weight of the total carbohydrate component. In addition, 2′-FL and/or LNnT may comprise (individually or together) about 0.1% to about 1.5% by weight of the total formula (e.g., from about 0.5% to about 1.0% by weight of the total formula).

In these or other embodiments, the infant formula has a fats/oils component of from about 15% to about 50% (i.e., 15 to 50 g fats/oils per 100 g of dry formula), such as from about 20% to about 40%, or about 20% to about 30%. Fats can include about 20% to about 50% (e.g., about 25% or about 30%) saturated fatty acids (e.g., butyric acid, capric acid, lauric acid, myristic acid, palmitic acid, and stearic acid), and from about 30% to about 50% monounsaturated fatty acids (e.g., palmitoleic acid (16:1), oleic acid (18:1)), and from about 5% to about 30% (e.g., about 20% or about 25%) polyunsaturated fatty acid (linoleic acid (18:2), linolenic acid (18:3), and/or arachidonic acid (20:4)). In some embodiments, the formula comprises one or more omega-3 fatty acids (e.g., DHA or EPA). In some embodiments, the formula comprises (in addition to one or more omega-3 fatty acids) an omega-6 fatty acid (e.g., arachidonic acid).

The fat sources for the fat component of the infant formula may be any of those known in the art, including but not limited to: animal sources such as milk fat, butter, butter fat, egg yolk lipid; marine sources, such as fish oils, marine oils, single cell oils; vegetable and plant oils, such as corn oil, canola oil, sunflower oil, soybean oil, palm oil, oil, palm olein oil, coconut oil, high oleic sunflower oil, safflower oil, high-oleic safflower oil, evening primrose oil, rapeseed oil, low erucic acid rapeseed oil (canola oil), olive oil, flaxseed (linseed) oil, cottonseed oil, high oleic safflower oil, palm stearin oil, palm kernel oil, wheat germ oil; medium chain triglyceride oils and emulsions and esters of fatty acids and SN2 palmitate oil; and any combinations thereof. In some embodiments, the fat sources are selected to avoid odors or flavors that are likely to be rejected by infants.

Milk fat globule membrane (MFGM) is a complex structure present in human and bovine milk and contains a broad variety of integral and peripheral proteins, glycoproteins enzymes, and lipids with antimicrobial and antiviral effects, that combat gut-derived infections. In some embodiments, the fat component comprises innate milk fat globule membrane (MFGM), added MFGM (e.g., isolated from human or animal milk), phospholipids, cholesterol, oil, non-hexane extracted docosahexaenoic acid (DHA), hexane extracted arachidonic acid (AA), non-hexane extracted AA, or a combination thereof. In certain embodiments, the oil comprises vegetable oil, soy oil, palm oil, or a combination thereof.

In various embodiments, the infant formula may comprise various vitamins and minerals. The selection and amount of vitamins and minerals will be according to the recommendation for each age group indicated for the formulation.

In other aspects, the invention provides a method for providing nutrition to a newborn or infant comprising feeding the newborn or infant with the infant formula disclosed herein. For example, the dry formula will be reconstituted with (i.e., solubilized in) water prior to feeding. In various embodiments, the infant is 0-6 months of age, or 6 to 12 months of age, or over 1 year of age (e.g., 1-2 years of age). In some embodiments, the newborn or infant is diagnosed with cow's milk protein allergy or allergic proctocolitis, and may be intolerant of EHF or AAF.

As used herein, unless the context requires otherwise, the term “about” means±10% of an associated number.

### EXAMPLES

### Example 1: Extraction of Human Milk Proteins, and Supplementation of Infant Formulas

In this example, human breast milk proteins were extracted, and used to supplement infant formulas. These studies demonstrate that superior infant formulas can be created with human protein supplementation.

Human breast milk proteins were extracted from human milk. The following general process was employed. Human milk was collected from healthy women and stored at −20° C. Before extraction, the samples were defrosted at room temperature and 15 mL of acetic acid was added to a total volume of 100 mL of breast milk, to acidify and precipitate casein. To remove fat and other particles, the breast milk was centrifuged 3 times at 1,500 rpm, for 15 minutes. Target milk proteins were then separated using membrane filters with the desired cutoff values. Here, proteins in the range of 10 to 50 kDa were recovered. For example, the liquid is placed on a membrane filter with molecular weight cutoff of 50 kD. The permeate is collected after 12×g centrifuge, for 60 minutes, which contains all proteins and molecules with less than 50 kD. All of this volume was placed on a further membrane filter having molecular weight cutoff of 10 kD, in order to retain proteins with values greater than this cutoff. These include α-lactalbumin (molecular weight of about 14 kD), osteopontin (molecular weight of about 33 kDa), and lysozyme (molecular weight of about 15 kDa), among others. After centrifugation at 12×g, for 60 minutes, the retentate was dialyzed, oven-dried, powdered and sifted.

This concentrated protein from extraction was used as an infant formula ingredient, along with lactose, vegetable oils (e.g., canola oil, coconut oil, sunflower oil), vitamins and minerals premix. The result was superior to commercial hypoallergenic infant formulas (casein hydrolysate and amino acid-based formulas) regarding flavor, odor, color, and general appearance. We conclude that infant formula containing extracted human breast milk proteins has better sensory characteristics as compared to available hypoallergenic formulas. Physical-chemical analysis further demonstrate the adequacy of the final product to nutritional recommendations.

Provided with this result, we conclude that it is possible to produce superior infant formulas based on supplementation with extracted or recombinant human breast milk proteins. Further, such results may be achieved with whey protein alone.

### Example 2: Exemplary Formulations Based on Recombinant or Extracted Proteins

The following example illustrates an infant formula containing a protein component based on recombinant or extracted human alpha-lactalbumin, enriched with amino acids, DHA, ARA and nucleotides. The following formula meets the needs of infants in the range of 0 to 12 months.
Energy distribution: Protein 8%, Carbohydrate 45%, Fat 47%.
Macronutrient distribution: Protein 9.5%, Carbohydrate 54%, Fat 25%.
Guidelines, preparation and use: 14.4 g of powder with 90 mL of water (100 mL total volume).

NUTRIENTS

| | | Per | Per | Per |
|---|---|---|---|---|
| Nutrients | Unit | 100 g | 100 ml | 100 kcal |
| Energy | kcal | 479 | 70 | 100 |
| Carbohydrate | g | 54 | 7.5 | 11.3 |
| Human breast milk proteins | g | 9.5 | 1.4 | 2.01 |
| α-lactalbumin | g | 9.5 | 1.4 | 2.01 |
| Fat | g | 25 | 3.5 | 5.3 |
| saturated fat | g | 7.4 | 1.0 | 1.54 |
| linoleic acid | mg | 4170 | 600 | 880 |
| α-linolenic acid | mg | 1040 | 150 | 220 |
| DHA | mg | 50 | 7.20 | 10.6 |
| ARA | mg | 87.5 | 12.6 | 18.5 |
| trans Fat | g | 0 | 0.0 | 0.0 |
| Fiber | g | 0 | 0.0 | 0.0 |
| Sodium | mg | 119 | 17.1 | 25.2 |
| Calcium | mg | 380 | 54.7 | 80 |
| Iron | mg | 8.5 | 1.22 | 1.8 |
| Chloride | mg | 308 | 44 | 65 |
| Potassium | mg | 500 | 72 | 106 |
| Phosphorus | mg | 255 | 37 | 53 |
| Magnesium | mg | 45 | 6.4 | 9.5 |
| Iodine | mcg | 92 | 13 | 19 |
| Copper | mcg | 403 | 58 | 85 |
| Zinc | mg | 3.5 | 0.5 | 0.7 |
| Manganese | mcg | 100 | 14 | 21 |
| Selenium | mcg | 12 | 1.07 | 2.51 |
| Vitamin A | mcg | 450 | 65 | 94 |
| Vitamin D | mcg | 9.4 | 1.35 | 1.96 |
| Vitamin E | mg | 8.0 | 1.2 | 1.7 |
| Vitamin K | mcg | 43 | 6.2 | 9.0 |
| Vitamin C | mg | 62 | 9.1 | 13 |
| Vitamin B1 | mcg | 380 | 55 | 79 |
| Vitamin B2 | mcg | 710 | 102 | 148 |
| Niacin | mcg | 4730 | 681 | 987 |
| Vitamin B6 | mcg | 380 | 55 | 79 |
| Folic acid | mcg | 71 | 10 | 15 |
| Pantothenic acid | mcg | 2130 | 307 | 450 |
| B12 vitamin | mcg | 1.4 | 0.2 | 0.29 |
| Biotin | mcg | 16 | 2.3 | 3.4 |
| Choline | mg | 115 | 16.6 | 24 |
| Inositol | mg | 27 | 3.9 | 5.6 |
| L-carnitine | mg | 8.0 | 1.2 | 1.7 |
| Taurine | mg | 44 | 6.3 | 9.0 |
| L-methionine | mg | 38 | 5.5 | 7.9 |
| L-phenylalanine | mg | 38 | 5.5 | 7.9 |
| L-valine | mg | 67 | 9.5 | 14 |
| Nucleotides | mg | 24.5 | 3.5 | 5.0 |

Ingredients: Lactose, vegetable oils (canola oil, coconut oil, sunflower oil), recombinant or extracted human milk alpha-lactoalbumin, and less than 2% Mortierella alpina* oil, Crypthecodinium cohnii** oil, calcium phosphate, potassium citrate, sodium chloride, potassium chloride, ferrous sulfate, magnesium phosphate, zinc sulfate, copper sulfate, manganese sulfate, potassium iodide, sodium selenite, soy lecithin, choline bitartrate, ascorbic acid, niacinamide, calcium pantothenate, riboflavin, thiamin hydrochloride, vitamin D3, pyridoxine hydrochloride, folic acid, vitamin K1, biotin, vitamin B12, inositol, vitamin E acetate, vitamin A palmitate, nucleotides (cytidine 5′-monophosphate, disodium uridine 5′-monophosphate, adenosine 5′-monophosphate, disodium guanosine 5′-monophosphate), L-valine, L-methionine, L-phenylalanine, taurine, L-carnitine.
*A source of arachidonic acid (ARA)
**A source of docosahexaenoic acid (DHA)

The following example illustrates an infant formula containing a protein component based on recombinant or extracted human serum albumin, enriched with amino acids, DHA, ARA and nucleotides. The following formula meets the needs of infants in the range of 0 to 12 months.
Energy distribution: Protein 8%, Carbohydrate 45%, Fat 47%.
Macronutrient distribution: Protein 10%, Carbohydrate 54%, Fat 25%.
Guidelines, preparation and use: 14.4 g of powder with 90 mL of water (100 mL total volume).

NUTRIENTS

| | | Per | Per | Per |
|---|---|---|---|---|
| Nutrients | Unit | 100 g | 100 ml | 100 kcal |
| Energy | kcal | 480 | 70 | 100 |
| Carbohydrate | g | 54 | 7.5 | 11.3 |
| Human breast milk proteins | g | 10 | 1.4 | 2.01 |
| Human serum albumin | g | 10 | 1.4 | 2.01 |
| Fat | g | 25 | 3.5 | 5.3 |
| saturated fat | g | 7.4 | 1.0 | 1.54 |
| linoleic acid | mg | 4170 | 600 | 880 |
| α-linolenic acid | mg | 1040 | 150 | 220 |
| DHA | mg | 50 | 7.20 | 10.6 |
| ARA | mg | 87.5 | 12.6 | 18.5 |
| trans Fat | g | 0 | 0.0 | 0.0 |
| Fiber | g | 0 | 0.0 | 0.0 |
| Sodium | mg | 119 | 17.1 | 25.2 |
| Calcium | mg | 380 | 54.7 | 80 |
| Iron | mg | 8.5 | 1.22 | 1.8 |
| Chloride | mg | 308 | 44 | 65 |
| Potassium | mg | 500 | 72 | 106 |
| Phosphorus | mg | 255 | 37 | 53 |
| Magnesium | mg | 45 | 6.4 | 9.5 |
| Iodine | mcg | 92 | 13 | 19 |
| Copper | mcg | 403 | 58 | 85 |
| Zinc | mg | 3.5 | 0.5 | 0.7 |
| Manganese | mcg | 100 | 14 | 21 |
| Selenium | mcg | 12 | 1.07 | 2.51 |
| Vitamin A | mcg | 450 | 65 | 94 |
| Vitamin D | mcg | 9.4 | 1.35 | 1.96 |
| Vitamin E | mg | 8.0 | 1.2 | 1.7 |
| Vitamin K | mcg | 43 | 6.2 | 9.0 |
| Vitamin C | mg | 62 | 9.1 | 13 |
| Vitamin B1 | mcg | 380 | 55 | 79 |
| Vitamin B2 | mcg | 710 | 102 | 148 |
| Niacin | mcg | 4730 | 681 | 987 |
| Vitamin B6 | mcg | 380 | 55 | 79 |
| Folic acid | mcg | 71 | 10 | 15 |
| Pantothenic acid | mcg | 2130 | 307 | 450 |
| B12 vitamin | mcg | 1.4 | 0.2 | 0.29 |
| Biotin | mcg | 16 | 2.3 | 3.4 |
| Choline | mg | 115 | 16.6 | 24 |
| Inositol | mg | 27 | 3.9 | 5.6 |
| L-carnitine | mg | 8.0 | 1.2 | 1.7 |
| Taurine | mg | 44 | 6.3 | 9.0 |
| L-isoleucine | mg | 357 | 51 | 75 |
| L-methionine | mg | 29 | 4.1 | 5.9 |
| L-threonine | mg | 32 | 4.6 | 6.7 |
| L-tryptophan | mg | 133 | 20 | 28 |
| Nucleotides | mg | 24.5 | 3.5 | 5.0 |

Ingredients: Lactose, vegetable oils (canola oil, coconut oil, sunflower oil), recombinant or extracted human serum albumin, and less than 2% Mortierella alpina* oil, Crypthecodinium cohnii** oil, calcium phosphate, potassium citrate, sodium chloride, potassium chloride, ferrous sulfate, magnesium phosphate, zinc sulfate, copper sulfate, manganese sulfate, potassium iodide, sodium selenite, soy lecithin, choline bitartrate, ascorbic acid, niacinamide, calcium pantothenate, riboflavin, thiamin hydrochloride, vitamin D3, pyridoxine hydrochloride, folic acid, vitamin K1, biotin, vitamin B12, inositol, vitamin E acetate, vitamin A palmitate, nucleotides (cytidine 5′-monophosphate, disodium uridine 5′-monophosphate, adenosine 5′-monophosphate, di sodium guanosine 5′-monophosphate), L-isoleucine, L-tryptophan, L-threonine, L-methionine, taurine, L-carnitine.
*A source of arachidonic acid (ARA)
**A source of docosahexaenoic acid (DHA)

The following example illustrates a formulation containing a protein component based on the recombinant or extracted human breast milk proteins α-lactoalbumin and β-casein, in a whey:casein ratio of 60:40, enriched with DHA, ARA and nucleotides. The following formula meets the needs of infants in the range of 0 to 12 months.
Energy distribution: Protein 8%, Carbohydrate 45%, Fat 47%.
Macronutrient distribution: Protein 9.5%, Carbohydrate 54%, Fat 25%.
Guidelines, preparation and use: 14.4 g of powder with 90 mL of water (100 mL total volume).

NUTRIENTS

| | | Per | Per | Per |
|---|---|---|---|---|
| Nutrients | Unit | 100 g | 100 ml | 100 kcal |
| Energy | kcal | 479 | 70 | 100 |
| Carbohydrate | g | 54 | 7.5 | 11.3 |
| Human breast milk proteins | g | 9.5 | 1.4 | 2.0 |
| α-lactalbumin | g | 5.7 | 0.84 | 1.19 |
| β-casein | g | 3.8 | 0.56 | 0.81 |
| Fat | g | 25 | 3.5 | 5.3 |
| saturated fat | g | 7.4 | 1.0 | 1.54 |
| linoleic acid | mg | 4170 | 600 | 880 |
| α-linolenic acid | mg | 1040 | 150 | 220 |
| DHA | mg | 50 | 7.20 | 10.6 |
| ARA | mg | 87.5 | 12.6 | 18.5 |
| trans Fat | g | 0 | 0.0 | 0.0 |
| Fiber | g | 0 | 0.0 | 0.0 |
| Sodium | mg | 119 | 17.1 | 25.2 |
| Calcium | mg | 380 | 54.7 | 80 |
| Iron | mg | 8.5 | 1.22 | 1.8 |
| Chloride | mg | 308 | 44 | 65 |
| Potassium | mg | 500 | 72 | 106 |
| Phosphorus | mg | 255 | 37 | 53 |
| Magnesium | mg | 45 | 6.4 | 9.5 |
| Iodine | mcg | 92 | 13 | 19 |
| Copper | mcg | 403 | 58 | 85 |
| Zinc | mg | 3.5 | 0.5 | 0.7 |
| Manganese | mcg | 100 | 14 | 21 |
| Selenium | mcg | 12 | 1.07 | 2.51 |
| Vitamin A | mcg | 450 | 65 | 94 |
| Vitamin D | mcg | 9.4 | 1.35 | 1.96 |
| Vitamin E | mg | 8.0 | 1.2 | 1.7 |
| Vitamin K | mcg | 43 | 6.2 | 9.0 |
| Vitamin C | mg | 62 | 9.1 | 13 |
| Vitamin B1 | mcg | 380 | 55 | 79 |
| Vitamin B2 | mcg | 710 | 102 | 148 |
| Niacin | mcg | 4730 | 681 | 987 |
| Vitamin B6 | mcg | 380 | 55 | 79 |
| Folic acid | mcg | 71 | 10 | 15 |
| Pantothenic acid | mcg | 2130 | 307 | 450 |
| B12 vitamin | mcg | 1.4 | 0.2 | 0.29 |
| Biotin | mcg | 16 | 2.3 | 3.4 |
| Choline | mg | 115 | 16.6 | 24 |
| Inositol | mg | 27 | 3.9 | 5.6 |
| L-carnitine | mg | 8.0 | 1.2 | 1.7 |
| Taurine | mg | 44 | 6.3 | 9.0 |
| Nucleotides | mg | 24.5 | 3.5 | 5.0 |

Ingredients: Lactose, vegetable oils (canola oil, coconut oil, sunflower oil), recombinant or extracted human milk alpha-lactoalbumin, recombinant human milk beta-casein and less than 2% Mortierella alpina oil, Crypthecodinium cohnii oil, calcium phosphate, potassium citrate, sodium chloride, potassium chloride, ferrous sulfate, magnesium phosphate, zinc sulfate, copper sulfate, manganese sulfate, potassium iodide, sodium selenite, soy lecithin, choline bitartrate, ascorbic acid, niacinamide, calcium pantothenate, riboflavin, thiamin hydrochloride, vitamin D3, pyridoxine hydrochloride, folic acid, vitamin K1, biotin, vitamin B12, inositol, vitamin E acetate, vitamin A palmitate, nucleotides (cytidine 5′-monophosphate, disodium uridine 5′-monophosphate, adenosine 5′-monophosphate, disodium guanosine 5′-monophosphate), taurine, L-carnitine.
*A source of arachidonic acid (ARA)
**A source of docosahexaenoic acid (DHA)

The following example illustrates a formulation containing the protein component based on the recombinant or extracted human breast milk proteins α-lactalbumin and β-casein, and enriched with lactoferrin and osteopontin, in a whey:casein ratio of 60:40. The formula is further enriched with MFGM, HMOs, DHA, ARA and nucleotides. The following formula meets the needs of infants in the range of 1 to 12 months.
Energy distribution: Protein 8%, Carbohydrate 45%, Fat 47%.
Macronutrient distribution: Protein 9.5%, Carbohydrate 54%, Fat 25%.
Guidelines, preparation and use: 14.4 g of powder with 90 mL of water (100 mL total volume).

NUTRIENTS

| | | Per | Per | Per |
|---|---|---|---|---|
| Nutrients | Unit | 100 g | 100 ml | 100 kcal |
| Energy | kcal | 479 | 70 | 100 |
| Carbohydrate | g | 54 | 7.5 | 11.3 |
| 2′-fucosyllactose | g | 0.8 | 0.12 | 0.17 |
| Human breast milk proteins | g | 9.5 | 1.4 | 2.0 |
| α-lactalbumin | g | 5.1 | 0.74 | 1.06 |
| β-casein | g | 3.8 | 0.56 | 0.80 |
| Lactoferrin | g | 0.50 | 0.07 | 0.10 |
| Osteopontin | g | 0.10 | 0.01 | 0.02 |
| Fat | g | 25 | 3.5 | 5.3 |
| saturated fat | g | 7.4 | 1.0 | 1.54 |
| linoleic acid | mg | 4170 | 600 | 880 |
| α-linolenic acid | mg | 1040 | 150 | 220 |
| DHA | mg | 50 | 7.20 | 10.6 |
| ARA | mg | 87.5 | 12.6 | 18.5 |
| trans Fat | g | 0 | 0.0 | 0.0 |
| Sodium | mg | 119 | 17.1 | 25.2 |
| Calcium | mg | 380 | 54.7 | 80 |
| Iron | mg | 8.5 | 1.22 | 1.8 |
| Chloride | mg | 308 | 44 | 65 |
| Potassium | mg | 500 | 72 | 106 |
| Phosphorus | mg | 255 | 37 | 53 |
| Magnesium | mg | 45 | 6.4 | 9.5 |
| Iodine | mcg | 92 | 13 | 19 |
| Copper | mcg | 403 | 58 | 85 |
| Zinc | mg | 3.5 | 0.5 | 0.7 |
| Manganese | mcg | 100 | 14 | 21 |
| Selenium | mcg | 12 | 1.07 | 2.51 |
| Vitamin A | mcg | 450 | 65 | 94 |
| Vitamin D | mcg | 9.4 | 1.35 | 1.96 |
| Vitamin E | mg | 8.0 | 1.2 | 1.7 |
| Vitamin K | mcg | 43 | 6.2 | 9.0 |
| Vitamin C | mg | 62 | 9.1 | 13 |
| Vitamin B1 | mcg | 380 | 55 | 79 |
| Vitamin B2 | mcg | 710 | 102 | 148 |
| Niacin | mcg | 4730 | 681 | 987 |
| Vitamin B6 | mcg | 380 | 55 | 79 |
| Folic acid | mcg | 71 | 10 | 15 |
| Pantothenic acid | mcg | 2130 | 307 | 450 |
| B12 vitamin | mcg | 1.4 | 0.2 | 0.29 |
| Biotin | mcg | 16 | 2.3 | 3.4 |
| Choline | mg | 115 | 16.6 | 24 |
| Inositol | mg | 27 | 3.9 | 5.6 |
| L-carnitine | mg | 8.0 | 1.2 | 1.7 |
| Taurine | mg | 44 | 6.3 | 9.0 |
| Nucleotides | mg | 24.5 | 3.5 | 5.0 |

Ingredients: Lactose, vegetable oils (canola oil, coconut oil, sunflower oil, SN2 palmitate oil), recombinant human milk alpha-lactoalbumin, recombinant human milk beta-casein, whey protein-lipid concentrate^{#} and less than 2% recombinant human milk lactoferrin, recombinant human milk osteopontin, 2′-fucosyllactose, Mortierella alpina* oil, Crypthecodinium cohnii** oil, calcium phosphate, potassium citrate, sodium chloride, potassium chloride, ferrous sulfate, magnesium phosphate, zinc sulfate, copper sulfate, manganese sulfate, potassium iodide, sodium selenite, soy lecithin, choline bitartrate, ascorbic acid, niacinamide, calcium pantothenate, riboflavin, thiamin hydrochloride, vitamin D3, pyridoxine hydrochloride, folic acid, vitamin K1, biotin, vitamin B12, inositol, vitamin E acetate, vitamin A palmitate, nucleotides (cytidine 5′-monophosphate, disodium uridine 5′-monophosphate, adenosine 5′-monophosphate, disodium guanosine 5′-monophosphate), taurine, L-carnitine.
#A source of MFGM
*A source of arachidonic acid (ARA)
**A source of docosahexaenoic acid (DHA)

### Example 3: Formulations Based on Nutrient Composition Variation During Lactation Period

The following example illustrates different formulations having a nutrient composition ranging their concentration, mimicking the variation observed in human milk during the lactation period.

Formulation 1 is intended for use by infants from birth until 10 days; formulation 2 is intended for use from 11 to 30 days; formulation 3 is intended for use from 31 to 90 days, and formulation 4 is intended for use from 3 months old and above.

| | Formulation 1 | | Formulation 2 | | Formulation 3 | | Formulation 4 | |
|---|---|---|---|---|---|---|---|---|
| | Per | Per | Per | Per | Per | Per | Per | Per |
| Nutrients | 100 g | 100 mL | 100 g | 100 mL | 100 g | 100 mL | 100 g | 100 mL |
| Energy | 491 | 71 | 482 | 69 | 480 | 69 | 479 | 69 |
| Carbohydrate | 52 | 7.5 | 53 | 7.6 | 54 | 7.8 | 54 | 7.8 |
| 2′-fucosyllactose | 1.0 | 0.14 | 1.0 | 0.14 | 0.8 | 0.12 | 0.8 | 0.12 |
| Human breast milk | 14.5 | 2.07 | 11.1 | 1.59 | 9.7 | 1.38 | 9.5 | 1.4 |
| proteins | | | | | | | | |
| α-lactalbumin | 2.45 | 0.35 | 3.36 | 0.48 | 2.73 | 0.39 | 2.31 | 0.33 |
| Lactoferrin | 2.45 | 0.35 | 2.24 | 0.32 | 1.26 | 0.18 | 1.19 | 0.17 |
| Serum albumin | 0.35 | 0.05 | 0.35 | 0.05 | 0.49 | 0.07 | 0.42 | 0.06 |
| Immunoglobulins | 5.6 | 0.8 | 1.05 | 0.15 | 0.84 | 0.12 | 0.84 | 0.12 |
| β-casein | 3.64 | 0.52 | 4.13 | 0.59 | 4.41 | 0.63 | 4.69 | 0.67 |
| whey:casein | 75:25 | | 63:37 | | 55:45 | | 50:50 | |
| Fat | 25 | 3.5 | 25 | 3.5 | 25 | 3.5 | 25 | 3.5 |
| saturated fat | 7.4 | 1.0 | 7.4 | 1.0 | 7.4 | 1.0 | 7.4 | 1.0 |
| linoleic acid | 4170 | 600 | 4170 | 600 | 4170 | 600 | 4170 | 600 |
| α-linolenic acid | 1040 | 150 | 1040 | 150 | 1040 | 150 | 1040 | 150 |
| DHA | 70 | 10.1 | 70 | 7.20 | 50 | 7.20 | 50 | 7.20 |
| ARA | 122 | 17.6 | 122 | 17.6 | 87.5 | 12.6 | 87.5 | 12.6 |
| Sodium | 119 | 17.1 | 119 | 17.1 | 250 | 36 | 250 | 36 |
| Calcium | 490 | 71 | 490 | 71 | 380 | 54.7 | 380 | 54.7 |
| Iron | 9.5 | 1.37 | 9.5 | 1.37 | 8.5 | 1.22 | 8.5 | 1.22 |
| Chloride | 308 | 44 | 308 | 44 | 440 | 63 | 440 | 63 |
| Potassium | 500 | 72 | 500 | 72 | 760 | 110 | 760 | 110 |
| Phosphorus | 255 | 37 | 255 | 37 | 255 | 37 | 255 | 37 |
| Magnesium | 45 | 6.4 | 45 | 6.4 | 45 | 6.4 | 45 | 6.4 |
| Iodine | 92 | 13 | 92 | 13 | 92 | 13 | 92 | 13 |
| Copper | 403 | 58 | 403 | 58 | 403 | 58 | 403 | 58 |
| Zinc | 4.2 | 0.6 | 3.5 | 0.5 | 3.5 | 0.5 | 3.5 | 0.5 |
| Manganese | 100 | 14 | 100 | 14 | 100 | 14 | 100 | 14 |
| Selenium | 14.4 | 2.07 | 12 | 1.07 | 12 | 1.07 | 12 | 1.07 |
| Vitamin A | 520 | 75 | 450 | 65 | 450 | 65 | 450 | 65 |
| Vitamin D | 12 | 1.7 | 9.4 | 1.35 | 9.4 | 1.35 | 9.4 | 1.35 |
| Vitamin E | 10 | 1.4 | 8.0 | 1.2 | 8.0 | 1.2 | 8.0 | 1.2 |
| Vitamin K | 43 | 6.2 | 43 | 6.2 | 43 | 6.2 | 43 | 6.2 |
| Vitamin C | 84 | 12.1 | 84 | 12.1 | 62 | 9.1 | 62 | 9.1 |
| Vitamin B1 | 350 | 50 | 350 | 50 | 380 | 55 | 380 | 55 |
| Vitamin B2 | 710 | 102 | 710 | 102 | 710 | 102 | 710 | 102 |
| Niacin | 4530 | 653 | 4530 | 653 | 4730 | 681 | 4730 | 681 |
| Vitamin B6 | 350 | 50 | 350 | 50 | 380 | 55 | 380 | 55 |
| Folic acid | 65 | 9 | 71 | 10 | 71 | 10 | 71 | 10 |
| Pantothenic acid | 1950 | 281 | 1950 | 281 | 2130 | 307 | 2130 | 307 |
| B12 vitamin | 1.8 | 0.26 | 1.8 | 0.26 | 1.4 | 0.2 | 1.4 | 0.2 |
| Biotin | 15 | 2.2 | 15 | 2.2 | 16 | 2.3 | 16 | 2.3 |
| Choline | 95 | 13.7 | 115 | 16.6 | 115 | 16.6 | 115 | 16.6 |
| Inositol | 27 | 3.9 | 27 | 3.9 | 27 | 3.9 | 27 | 3.9 |
| L-carnitine | 8.0 | 1.2 | 8.0 | 1.2 | 8.0 | 1.2 | 8.0 | 1.2 |
| Taurine | 44 | 6.3 | 44 | 6.3 | 44 | 6.3 | 44 | 6.3 |
| Nucleotides | 24.5 | 3.5 | 24.5 | 3.5 | 24.5 | 3.5 | 24.5 | 3.5 |

Ingredients: Lactose, vegetable oils (canola oil, coconut oil, sunflower oil, SN2 palmitate oil), recombinant human milk beta-casein, recombinant human milk alpha-lactalbumin, recombinant human milk lactoferrin, recombinant human milk immunoglobulin, and less than 2% recombinant human serum albumin, 2′-fucosyllactose, Mortierella alpina* oil, Crypthecodinium cohnii** oil, calcium phosphate, potassium citrate, sodium chloride, potassium chloride, ferrous sulfate, magnesium phosphate, zinc sulfate, copper sulfate, manganese sulfate, potassium iodide, sodium selenite, soy lecithin, choline bitartrate, ascorbic acid, niacinamide, calcium pantothenate, riboflavin, thiamin hydrochloride, vitamin D3, pyridoxine hydrochloride, folic acid, vitamin 1(1, biotin, vitamin B12, inositol, vitamin E acetate, vitamin A palmitate, nucleotides (cytidine 5′-monophosphate, disodium uridine 5′-monophosphate, adenosine 5′-monophosphate, di sodium guanosine 5′-monophosphate), taurine, L-carnitine.

### REFERENCES

AFFOLTER, M., et al. Temporal changes of protein composition in breast milk of Chinese urban mothers and impact of caesarean section delivery. Nutrients, 8 (504), 1-15, 2016.
AKHTAR ALI, S. et al. Nutritional hypophosphatemic rickets secondary to Neocate® use. Osteoporosis International, v. 30, n. 9, p. 1887-1891, 2019.
BOCQUET, A. et al. Efficacy and safety of hydrolyzed rice-protein formulas for the treatment of cow's milk protein allergy. Archives de Pediatrie, v. 26, n. 4, p. 238-246, 2019.
CANTANI, A.; MICERA, M. Immunogenicity of hydrolysate formulas in children (part 1). Analysis of 202 reactions. Journal of Investigational Allergology and Clinical Immunology, v. 10, n. 5, p. 261-276, 2015.
DUPONT, C. et al. Safety of a new amino acid formula in infants allergic to cow's milk and intolerant to hydrolysates. Journal of Pediatric Gastroenterology and Nutrition, v. 61, n. 4, p. 456-463, 2015.
GONZALEZ BALLESTEROS, L. F. et al. Unexpected widespread hypophosphatemia and bone disease associated with elemental formula use in infants and children. Bone, v. 97, p. 287-292, 2017.
GUO, MINGRUO. 2021. Human Milk Biochemistry and Infant Formula Manufacturing Technilogy. 2nd ed. Woodhead Publishing.
INUO, C. et al. Oral immunotherapy using partially hydrolyzed formula for cow's milk protein allergy: A randomized, controlled trial. International Archives of Allergy and Immunology, v. 177, n. 3, p. 259-268, 2018.
KOLETZKO, S. et al. Diagnostic approach and management of cow's-milk protein allergy in infants and children: Espghan gi committee practical guidelines. Journal of Pediatric Gastroenterology and Nutrition, v. 55, n. 2, p. 221-229, 2012.
LAYMAN, D. K., et al. Applications for α-lactalbumin in human nutrition. Nutrition Reviews, 76 (6), 444-460, 2018.
LONNERDAL, B. Nutritional and Physiologic Significance of Human Milk Proteins. The American Journal of Clinical Nutrition 77(6), 2003.
MARTIN, V. M. et al. Prospective Assessment of Pediatrician-Diagnosed Food Protein—Induced Allergic Proctocolitis by Gross or Occult Blood. Journal of Allergy and Clinical Immunology: In Practice, v. 8, n. 5, p. 1692-1699.el, 2020.
MIRAGLIA DEL GIUDICE, M. et al. Flavor, relative palatability and components of cow's milk hydrolysed formulas and amino acid-based formula Allergology and Immunology. Italian Journal of Pediatrics, v. 41, n. 1, 2015.
PEDROSA DELGADO, M. et al. Palatability of hydrolysates and other substitution formulas for cow's milk-allergic children: A comparative study of taste, smell, and texture evaluated by healthy volunteers. Journal of Investigational Allergology and Clinical Immunology, v. 16, n. 6, p. 351-356, 2006.
PERRIN, MARYANNE T., et al. A Longitudinal Study of Human Milk Composition in the Second Year Postpartum: Implications for Human Milk Banking. Maternal and Child Nutrition 13(1):1-12, 2017.
SALA-VILA, ALEIX, et al. Lipid Composition in Human Breast Milk from Granada (Spain): Changes during Lactation. Nutrition 21(4):467-73, 2005.
SINKIEWICZ-DAROL, ELENA, et al. Nutrients and Bioactive Components of Human Milk After One Year of Lactation. Implication for Human Milk Banks. Journal of Pediatric Gastroenterology & Nutrition Publish Ahead of Print:87-100, 2021.
VANDENPLAS, Y.; DE GREEF, E.; DEVREKER, T. Treatment of cow's milk protein allergy. Pediatric Gastroenterology, Hepatology and Nutrition, v. 17, n. 1, p. 1-5, 2014.
VANDERHOOF, J.; MOORE, N.; DE BOISSIEU, D. Evaluation of an amino acid-based formula in infants not responding to extensively hydrolyzed protein formula. Journal of Pediatric Gastroenterology and Nutrition, 2016.
YUAN, TINGLAN, et al. Medium- and Long-Chain Triacylglycerols Composition in Preterm and Full-Term Human Milk across Different Lactation Stages. Lwt 142 (December 2020):110907, 2021.
ZOU, XIAO QIANG, et al. Human Milk Fat Globules from Different Stages of Lactation: A Lipid Composition Analysis and Microstructure Characterization. Journal of Agricultural and Food Chemistry 60(29):7158-67, 2012.

1. An infant formula comprising recombinant or extracted human milk proteins, and nutrients selected from oils, carbohydrates, amino acids selected from essential and non-essential amino acids, vitamins, minerals, and nitrogen sources.

2. The formula of claim 1, wherein the formula does not contain any non-human animal protein.

3. The formula of claim 2, wherein the formula does not comprise plant protein.

4. The formula of claim 3, wherein the formula does not comprise hydrolyzed milk protein.

5. The formula of any one of claims 1 to 4, wherein the formula comprises about 15 recombinant human breast milk proteins or less.

6. The formula of claim 5, wherein the formula comprises about 12 recombinant human breast proteins or less.

7. The formula of claim 5, wherein the formula comprises about 10 recombinant human breast milk proteins or less, or about 8 recombinant human breast milk proteins or less, or about 5 recombinant human breast milk proteins or less.

8. The formula of claim 7, wherein the formula comprises from 2 to 7 recombinant human breast milk proteins, or from 3 to 5 recombinant human breast milk proteins.

9. The formula of claim 5, wherein the formula comprises only human whey proteins without caseins.

10. The formula of claim 5, wherein the formula comprises from 1 to 5 recombinant human whey proteins with human beta or kappa casein.

11. The formula of claim 8, wherein the recombinant human breast milk proteins are selected from α-lactalbumin, β-caseins, κ-Casein, lactoferrin, osteopontin, serum albumin, lysozyme, immunoglobulins (IgA) and Epidermal Growth Factors (EGF).

12. The formula of claim 11, wherein the formula contains at least three recombinant human breast milk proteins selected from a α-lactalbumin, β-casein, κ-casein, lactoferrin, osteopontin, serum albumin, lysozyme, Immunoglobulin (e.g., IgA) and Epidermal Growth Factor (EGF).

13. The formula of claim 11, wherein the human protein component contains from about 1% to about 100% recombinant human casein(s), which can be selected from β caseins and/or κ caseins.

14. The formula of claim 13, wherein the human protein component contains from about 5% to about 75% recombinant human casein(s), which can be selected from β caseins and/or κ caseins.

15. The formula of claim 14, wherein the human protein component contains from about 5% to about 50% recombinant human casein(s), which can be selected from β caseins and/or κ caseins.

16. The formula of claim 14, wherein the human protein component contains from about 20% to about 50% recombinant human casein(s), or from about 20% to about 40% recombinant human caseins, which are optionally selected from β caseins and/or κ caseins.

17. The formula of claim 7, wherein the human protein component is from about 1% to about 100% recombinant human α-lactalbumin.

18. The formula of claim 17, wherein the human protein component is from about 5% to about 75% recombinant human α-lactalbumin, or from about 5% to about 50% recombinant human α-lactalbumin, or about 5% to about 40% recombinant human α-lactalbumin, or from about 5% to about 30% recombinant human α-lactalbumin.

19. The formula of claim 17, wherein the human protein component is from about 50% to about 100% recombinant human α-lactalbumin, or from about 50% to about 90% recombinant human α-lactalbumin, or from about 50% to about 75% recombinant human α-lactalbumin.

20. The formula of claim 17, wherein recombinant human α-lactalbumin is the only protein in the formulation.

21. The formula of claim 20, wherein the formula is supplemented with free amino acids, which are optionally essential amino acids.

22. The formula of claim 21, wherein the formula is supplemented with methionine.

23. The formula of claim 22, wherein the formula is supplemented with phenylalanine and valine.

24. The formula of any one of claims 7 to 9, wherein the human protein component is from about 1% to about 100% recombinant human lactoferrin, or from about 1% to about 75% recombinant human lactoferrin, or from about 1% to about 50% recombinant human lactoferrin, or about 1% to 40% recombinant human lactoferrin, or from about 1% to about 40% recombinant human lactoferrin, or from about 1% to about 10% recombinant human lactoferrin.

25. The formula of claim 24, wherein the protein component is from about 50% to about 100% recombinant human lactoferrin, or from about 50% to about 90% recombinant human lactoferrin, or from about 50% to about 75% recombinant human lactoferrin.

26. The formula of any one of claims 7 to 9, wherein the human protein component is from about 10% to about 100% recombinant human albumin, or from about 10% to about 75% recombinant human albumin, or from about 10% to about 50% recombinant human albumin, or about 10% to 40% recombinant human albumin, or from about 10% to about 30% recombinant human albumin.

27. The formula of claim 26, wherein the human protein component is from about 50% to about 100% recombinant human albumin, or from about 50% to about 90% recombinant human albumin, or from about 50% to about 75% recombinant human albumin, optionally where human albumin is the only protein in the formulation.

28. The formula of claim 26 or 27, wherein the formula is supplemented with isoleucine.

29. The formula of claim 28, wherein the formula is supplemented with tryptophan.

30. The formula of claim 28, wherein the formula is supplemented with threonine and/or methionine.

31. The formula of any one of claims 7 to 9, wherein the human protein component is from about 1% to about 100% recombinant human lysozyme, or from about 1% to about 75% recombinant human lysozyme, or from about 1% to about 50% recombinant human lysozyme, or about 1% to 40% recombinant human lysozyme, or from about 10% to about 40% recombinant human lysozyme.

32. The formula of claim 31, wherein the human protein component is from about 50% to about 100% recombinant human lysozyme, or from about 50% to about 90% recombinant human lysozyme, or from about 50% to about 75% recombinant human lysozyme.

33. The formula of any one of claims 7 to 9, wherein the human protein component is from about 5% to about 100% recombinant human IgA, or from about 5% to about 75% recombinant human IgA, or from about 5% to about 50% recombinant human IgA, or from about 5% to about 40% recombinant human IgA, or from about 10% to about 30% recombinant human IgA.

34. The formula of claim 33, wherein the human protein component is from about 50% to about 100% recombinant human IgA, or from about 50% to about 90% recombinant human IgA, or from about 50% to about 75% recombinant human IgA.

35. The formula of any one of claims 7 to 9, wherein the human protein component is about 0.001% to about 10% of recombinant human EGF, or from about 0.01% to about 5% recombinant human EGF, or from about 0.01% to about 1% recombinant human EGF, or from about 0.01% to about 0.1% recombinant human EGF.

36. The formula of any one of claims 1 to 4, comprising proteins extracted from human milk.

37. The formula of claim 36, wherein one or more whey proteins are extracted.

38. The formula of claim 36 or 37, wherein extracted proteins comprise one or more of α-lactalbumin, osteopontin, and lysozyme.

39. The formula of any one of claims 36 to 38, wherein extracted proteins include proteins having an apparent molecular weight of at least about 5 kDa, or at least about 10 kDa.

40. The formula of claim 39, wherein extracted proteins comprise proteins having an apparent molecular weight of less than about 100 kDa, or less than about 75 kDa, or less than about 50 kDa, or less than about 40 kDa.

41. The formula of any one of claims 1 to 40, wherein the recombinant or extracted human proteins comprise one or more of β-caseins, α-lactalbumin, lactoferrin, and osteopontin; and optionally EGF.

42. The formula of claim 41, wherein 100 g of the dry formula contains from about 5 g to about 15 g human recombinant or extracted human protein, and which is optionally consists of α-lactalbumin.

43. The formula of claim 41, wherein 100 g of the dry formula contains from about 3 g to about 10 g human recombinant or extracted human α-lactalbumin and from about 1 g to about 10 g of human recombinant or extracted β-casein.

44. The formula of claim 41, wherein 100 g of the dry formula contains from 2 g to about 5 g human recombinant β-caseins, about 3 g to about 8 g human recombinant α-lactalbumin, and about 0.1 g to about 3 g human recombinant lactoferrin, and optionally about 0.01 to about 1 g of osteopontin.

45. The formula of any one of claims 1 to 44, wherein the formula has a protein content of about 8 grams to 25 grams of protein per 100 grams of dry formula.

46. A set of at least three infant formulas comprising recombinant whey and casein proteins, the three infant formulas having a decreasing whey:casein ratio.

47. The set of infant formulas of claim 46, wherein a first infant formula intended for use in the first 1-2 weeks contains a whey:casein ratio of about 75:25; a second infant formula intended for use after the first formula and up to about the first month contains a whey:casein ratio of about 63:37; a third infant formula intended for use after the second formula and up to about 3 months contains a whey:casein ratio of about 55:45; and a fourth infant formula intended for use after the third formula contains a whey:casein ratio of about 50:50.

48. The formula of any one of claims 1 to 45, or the set of formulas of claim 46 or 47, wherein the formula has a carbohydrate component of from about 30 grams to about 50 grams per 100 grams of dry formula.

49. The formula of any one of claims 1 to 45 or the set of formulas of claim 46 or 47, wherein the formula has a fats and oils component of from about 30 grams to about 50 grams fats and oils per 100 grams of dry formula.

50. The formula or set of formulas of claim 49, wherein the fats and oils comprise saturated fatty acids optionally selected from butyric acid, capric acid, lauric acid, myristic acid, palmitic acid, and stearic acid.

51. The formula or set of formulas of claim 49 or 50, wherein the fats and oils comprise monounsaturated fatty acids optionally selected from palmitoleic acid and oleic acid.

52. The formula or set of formulas of any one of claims 49 to 51, wherein the fats and oils comprise polyunsaturated fatty acid optionally selected from linoleic acid, linolenic acid, and arachidonic acid.

53. The formula or set of formulas of claim 52, wherein the oils are vegetable oils selected from one or more of canola oil, coconut oil, sunflower oil, and corn oil.

54. The formula or set of formulas of any one of claims 49 to 53, wherein the fats and oils comprise one or more omega-3 fatty acids, optionally selected from DHA and EPA.

55. The formula or set of formulas of any one of claims 1 to 54, wherein the formula comprises amino acids or nitrogen sources optionally selected from choline, taurine, and carnitine.

56. The formula or set of formulas of any one of claims 1 to 55, wherein the formula comprises vitamins and nutrients optionally selected from Vitamin A, Vitamin D, Vitamin E, Vitamin K, Vitamin C, Vitamin B1, Vitamin B2, Niacin, Vitamin B6, folic acid, pantothenic acid, biotin, and nucleotides.

57. The formula or set of formulas of any one of claims 1 to 56, wherein the formula comprises minerals and/or salts optionally selected from sodium, calcium, iron, chlorine, potassium, phosphor, magnesium, iodine, copper, zinc, manganese, selenium, chromium, and molybdenum.

58. The formula or set of formulas of any one of claims 1 to 57, wherein the formula further comprises fibers, and optionally probiotics and/or prebiotics.

59. The formula or set of formulas of any one of claims 1 to 58, wherein the formula is a dry formula.

60. The formula or set of formulas of any one of claims 1 to 58, wherein the formula is provided constituted with water.

61. A method for providing nutrition to a newborn or infant comprising feeding the newborn or infant with the infant formula or set of formulas of any one of claims 1 to 60.

62. The method of claim 61, wherein the newborn or infant is diagnosed with cow's milk protein allergy or allergic proctocolitis.
